(19) United States
(12) Patent Application Publication
(10) Pub. No.: US 2010/0004194 A1
(43) Pub. Date: Jan. 7, 2010

(54) USE OF ISOMALTULOSE IN FOOD PRODUCTS HAVING A REGENERATIVE EFFECT

(76) Inventors: Aloys Berg, Waldkirch (DE); Daniel König, Freiburg (DE); Jörg Kowalczyk, Eisenberg/Steinborn (DE); Gunhild Kozianowski, Grunstadt (DE); Stephan Theis, Bad Durkheim (DE)

Correspondence Address: OSTROLENK FABER GERB & SOFFEN, 1180 AVENUE OF THE AMERICAS, NEW YORK, NY, 100368403, US

(21) Appl. No.: 12/375,737

(22) PCT Filed: Jul. 9, 2007
(86) PCT No.: PCT/EP2007/006079

§ 371 Date: Jan. 30, 2009

(30) Foreign Application Priority Data
Jul. 31, 2006 (DE) 10 2006 035 912.7

(51) Int. Cl. A61K 31/715; A61P 43/00

(52) U.S. Cl. 514/53

(57) ABSTRACT

The invention relates to the use of isomaltulose or mixtures of isomaltulose for the production of functional food products for improving the regeneration of individuals exposed to physical exertion.

The present invention relates to new uses of isomaltulose and to mixtures containing isomaltulose.

Current nutrition guidelines stipulate that at least 50% of the daily supply of energy should be provided in the form of carbohydrates. Low-glycaemic carbohydrates are in particular recommended in this regard, as these allow high blood glucose and insulin reactions to be avoided. With carbohydrates and especially glucose, muscles, the brain and nerves can operate equally well. Blood erythrocytes, the renal medulla and the nervous system are necessarily dependent on glucose as a source of energy. A carbohydrate deficiency in the metabolism owing to insufficient uptake of carbohydrates leads to hypoglycaemia, reduced glucose tolerance, ketosis and disturbances within the water and mineral balance. Maintaining the blood glucose level is important above all for the continuous supply to the brain and nerve cells and also to the blood cells of glucose as an energy source. A drawback is the body's relatively limited capacity to store carbohydrates. The storage form of carbohydrates is glycogen in the liver and muscles.

The glycogen stores are emptied also during relatively extensive physical or sporting uses. The greater the exertion intensity, the larger the proportion of carbohydrates in the provision of energy and the more the glycogen stores are emptied. This leads to disturbance of blood glucose homeostasis, and this also impairs the ability to concentrate and coordinate and leads to exhaustion. The primary nutritional aim is therefore to ensure, by way of carbohydrate-rich food, optimum glycogen stores and to have available as a source of energy, by consuming sufficient carbohydrates even during physical activity, as much carbohydrate as possible and also in relatively extensive activities and sporting uses.

In this connection, high fat oxidation is advantageous for providing energy during physical activities, as it saves the glycogen reserves, for example also for later exertion phases or the final spurt. It is generally known that training can increase fat oxidation at a specific exertion intensity. The better trained an individual is, the higher the fat oxidation. The aim of physical training is therefore inter alia in particular the optimisation and adaptation of the burning of fat. Increased fat oxidation is accordingly also a sign of successful training.

A high fat burn is furthermore advantageous for controlling weight and with regard to the prevention and treatment of overweight and secondary or concomitant disorders such as diabetes, impaired glucose tolerance, lipid metabolism disorders, arteriosclerosis, metabolic syndrome, diseases of the liver and other metabolic diseases. It is thus known that a low fat burn leads to a positive fat balance and an excess of metabolic energy and causes the formation of overweight or counteracts weight control and weight reduction. Strategies for preventing and treating overweight and secondary disorders therefore seek to increase fat burn and fat oxidation and to achieve a negative fat and energy balance.

On the other hand, it is however known that consumption of carbohydrates reduces fat oxidation, promotes carbohydrate oxidation and leads to a higher respiratory quotient. The respiratory quotient (RQ) reflects the ratio of CO₂/O₂ in the inhaled air and is a measure for which nutrients are burned. Pure carbohydrate burning leads to a respiratory quotient of 1, whereas pure fat burning leads to a respiratory quotient of 0.7. This adverse effect, i.e. the restriction of fat oxidation, continues for several hours after the carbohydrates have been consumed. Furthermore, it is known that fat oxidation continues to be reduced even in individuals who participate in sporting or physical exercise (Achten & Jeukendrup, 2003). Thus, on consumption of carbohydrates prior to physical exercise, the overall fat oxidation over an 8-hour period was reduced by approx. 30% (Schneiter et al., 1995). Furthermore, it is known that if carbohydrates are consumed during sporting/physical exercise, free fatty acids (FFAs) decrease more rapidly than if no carbohydrates are consumed. Fat oxidation is thus restricted by carbohydrates, as a changeover from fat oxidation to fat storage takes place more rapidly. This reduction of lipolysis and fat oxidation after carbohydrate consumption, which continues even beyond the end of the exertion, contributes to an excess of energy and to the promotion of fat deposition.

Comparing carbohydrate sources having a differing effect on the blood glucose and insulin level, lower-glycaemic carbohydrates are frequently advantageous. Lower-glycaemic carbohydrates would lead to a lower reduction of fat oxidation than higher-glycaemic carbohydrates. On the other hand, it is however known that fat oxidation is reduced after the consumption both of foods having a high GI (glycaemic index) and of foods having a low GI and that fat oxidation is lower if no carbohydrates at all are consumed (Brand-Miller et al., 2002). It is also known that fructose, a carbohydrate having a low GI and low insulin effect even causes a greater reduction of fat oxidation than glucose (Tittelbach et al., 2000). Consumption of carbohydrates thus leads generally to lower fat oxidation.

Finally, it is known that changes which occur in an individual's respiratory quotient during physical exercise are compensated for after the end of exertion. Thus, in the case of a higher respiratory quotient during high-intensive exertion, a lower respiratory quotient was observed in the post-exertion phase, whereas the respiratory quotient was lower during low-intensive exertion and higher after the end of exertion (Saris et al., 2004). Dionne et al. (1999) found that on consumption of a carbohydrate-containing drink immediately after physical activity, the RQ was not reduced owing to a compensatory effect. In particular, it is known that the consumption of high-glycaemic and low-glycaemic carbohydrates immediately after physical exertion leads to higher carbohydrate oxidation and thus to a higher RQ compared to oxidation prior to the start of exertion (Tittelbach et al., 2000). From Burke et al. (1998), it is also known that the glycaemic quality of a first meal, represented through the comparison of a meal having a high glycaemic index with a meal having a low glycaemic index, after consumption of further carbohydrates at a later point in time, i.e. after ingestion of a second meal, has no bearing on the respiratory quotient obtained thereafter, as an identical respiratory quotient was obtained in both cases.

However, it is in many cases desirable for food products ingested even after physical exertion not to lead to an increased RQ, in particular compared to the RQ prior to the start of exertion, but rather for a particularly low respiratory quotient to be present even after the end of physical exertion, even if carbohydrate-containing food products are consumed after the end of exertion. In particular, it is desirable if a low respiratory quotient reduced in this way remains reduced not only immediately after consumption of a meal, but rather in addition also after exertion and after consumption of a further, i.e. a second meal. With regard to a striven-for improved regeneration of the body tissue, in particular also with regard to the composition thereof, which should be as low-fat and glycogen-rich as possible, and the striven-for training effect, it is desirable to provide a low respiratory quotient even after completion of physical exertion and in particular also on consumption of food products after completion of physical exertion.

Although it is known that the consumption of specific carbohydrates, for example trehalose or isomaltulose, during physical exercise can lead to an increase in fat oxidation (WO 2005/013720), it has to date been assumed that such an increase in fat oxidation, caused by carbohydrates, also owing to the compensatory effect described hereinbefore, will not occur on consumption of carbohydrates after completion of physical exertion, but rather that an increase in the RQ is to be expected.

The present invention is therefore based on the technical problem of disclosing also to individuals who have just undergone physical exertion nutritional possibilities which ensure the desired training effect and the desired regeneration, in particular of their body composition. It is desirable, despite the uptake of carbohydrates in the post-exertion phase, in particular to optimise fat burn, to reduce the fat content in the body composition and to build up long-acting carbohydrate stores, in particular glycogen reserves. In particular, the present invention is also based on the technical problem of disclosing a teaching with which an advantageous reduced, lower respiratory quotient extends, i.e. is maintained in reduced form, not only after a first ingestion of food after the end of exertion, but rather in addition also over a subsequent second meal.

The present invention solves this technical problem by providing the teaching of using isomaltulose or mixtures of isomaltulose, in particular mixtures of isomaltulose with other carbohydrates, for the production of functional food products for improving the regeneration of individuals exposed to physical exertion. In a particularly advantageous manner, these functional food products are intended and suitable for consumption after, preferably immediately after, for example 0 to 24, 0 to 12, 0 to 4 or 0 to 2 hours after, conclusion of the physical exertion.

The term “immediately after, for example 0 hours” refers to the fact that the food products are ingested not at the same time as the physical exertion, but rather after physical exertion, in particular shortly thereafter, namely for example one minute or a few minutes up to for example 24 hours after the end of the physical exertion.

The term “regeneration” refers in the context of the present invention in particular to a regeneration of the body tissue, in particular of the composition of the body tissue, which can continue for a plurality of hours to days. Regeneration in the sense of the present invention is a build-up continuing beyond the end of exertion or after the exertion and a material fixing of carbohydrates which can be used for a relatively long period of time, i.e. polysaccharides, in particular glycogen, and the obtaining of a lipid metabolism based on fat, i.e. using fat, in particular a breakdown of fat contents in tissue. Regeneration in the sense of the present invention is a transformation and build-up of the body's fat and carbohydrate composition resulting, at the same time as a reduction of the fat content, in a build-up of carbohydrates which can be used for a relatively long period of time, in particular glycogen.

In the context of the present invention, improved regeneration refers in particular also to a transformation with a change in body composition leading to a reduced fat content and an increased glycogen content therein. Improved regeneration in the sense of the present invention is therefore a metabolic process leading to a higher glycogen content and a lower fat content in the composition of the body.

A clear distinction must be drawn between regeneration in the sense of the present invention and replenishing (what is known as “recovery”) of energy reserves which are available for a short period of time, for example glucose monosaccharides and ATP.

It has surprisingly been found that the consumption of isomaltulose and isomaltulose-containing mixtures after physical exertion leads to a particularly low respiratory quotient which is substantially equal to or even lower than immediately prior to the exertion; this was not to be expected in view of the prior art discussed hereinbefore. Conversely, the consumption of other carbohydrates after physical exertion, for example of maltodextrin, led to a respiratory quotient after physical exertion that was as much as 13% higher than prior to the exertion. Even when carbohydrate-free food products were consumed, i.e. in a placebo, the respiratory quotient after exertion was much higher, by about 6%, than before.

In a particularly preferred embodiment, it was found that the consumption of isomaltulose and isomaltulose-containing mixtures after physical exertion leads to an, as stated hereinbefore, particularly low respiratory quotient which surprisingly extends in this reduced form even well beyond the end of exertion, even beyond a second meal. The present invention therefore provides what is known as a second meal effect for isomaltulose. In connection with the present invention, a second meal effect refers to the fact that the respiratory quotient which occurs after the consumption of isomaltulose and exertion is much lower than in the case of a high-glycaemic food product and that this low respiratory quotient was still obtained, i.e. was maintained in this low form, in contrast to the high-glycaemic food products, even after consumption of a second meal.

In a particularly preferred embodiment, the present invention relates to the use of isomaltulose or mixtures of isomaltulose for the production of functional food products for providing a second meal effect, in particular in a preferred embodiment a second meal effect which is indicated by a respiratory quotient which is present after exertion and after a subsequent second ingestion of food and is substantially lower than or at least equal to a respiratory quotient of the same individual that is present immediately prior to a first ingestion of food and physical exertion.

According to the invention, a second meal effect refers to the fact that an RQ which occurs after a first meal remains substantially unaltered even after ingestion of a second meal, a second meal being a meal which is ingested subsequently to the first meal.

In a particularly preferred embodiment, the second meal, like the first meal, is a meal comprising functional food products of the present invention, i.e. isomaltulose or isomaltulose-containing mixtures. A further, likewise preferred embodiment provides for the second meal to be an isomaltulose-free or isomaltulose mixture-free meal.

A particularly preferred embodiment provides a use wherein the food products are suitable and intended to be consumed in the form of a first meal of two meals at different times after completion of the physical exertion and wherein the improved regeneration is indicated by a respiratory quotient which is present after exertion and after consumption of both meals and is substantially equal to or lower than a respiratory quotient of the same individual that is present immediately prior to the physical exertion and wherein the food products at least of the first meal, preferably of both meals are intended and suitable for consumption after completion of the physical exertion. A particularly preferred embodiment provides for the second meal to contain no isomaltulose or isomaltulose-containing mixtures, i.e. to be free from isomaltulose. Obviously, provision may however also be made for the second meal, like the first meal, to contain isomaltulose or isomaltulose-containing mixtures and thus to be a functional food product of the present invention.

In a preferred embodiment, the food products are suitable and intended to be consumed in the form of at least one meal, preferably two meals at different times or separated by a time-lag, after completion of the physical exertion.

In a further preferred embodiment, the present invention relates to the use of isomaltulose or mixtures thereof for the production of functional food products for providing a second meal effect which is indicated by a respiratory quotient which is present after a second ingestion of food and is substantially lower than or at least equal to a respiratory quotient of the same individual that is present immediately prior to a first ingestion of food.

In a preferred embodiment, the second ingestion of food is provided after the single or, if two exertions are provided, after the second physical exertion which lasts for example for 30 minutes.

The present invention relates in a particularly preferred embodiment to the use of isomaltulose or mixtures of isomaltulose for the production of functional food products for improving the regeneration of individuals exposed to physical exertion, wherein this improved regeneration is indicated by a respiratory quotient which is present after exertion and subsequent ingestion of food products and is preferably substantially equal to a respiratory quotient of the same individual that is present immediately prior to the physical exertion. Preferably, these food products are suitable and intended for consumption after completion of the physical exertion.

In a further preferred embodiment, the invention relates to the aforementioned use of isomaltulose or mixtures of isomaltulose for the production of functional food products, wherein the improved regeneration is indicated by a respiratory quotient which is present after exertion and subsequent ingestion of food products and is preferably substantially lower than a respiratory quotient of the same individual that is present immediately prior to the physical exertion. Preferably, these food products are suitable and intended for consumption after completion of the physical exertion.

In a further preferred embodiment, the present invention relates to the use of isomaltulose or mixtures of isomaltulose for the production of functional food products for improving the regeneration of individuals exposed to physical exertion, wherein the improved regeneration is indicated by a respiratory quotient which on use of isomaltulose or mixtures of isomaltulose is lower compared to the use of other carbohydrates in the otherwise identical food product, in particular compared to high-glycaemic carbohydrates, for example glucose, maltodextrin, glucose syrup or sucrose, in the same individual.

In a further preferred embodiment, the present invention relates to the use of isomaltulose or mixtures of isomaltulose for the production of functional food products for improving the regeneration of individuals exposed to physical exertion, wherein the improved regeneration is indicated by a respiratory quotient which on use of isomaltulose or mixtures of isomaltulose in the functional food product in the same individual is lower than if no carbohydrates are used in the otherwise identical functional food products.

A preferred embodiment of the present invention provides a use according to which the isomaltulose or mixtures containing isomaltulose also reduce(s) the rise of an RQ which is caused without the presence of isomaltulose or the mixture in an otherwise identical food product by the presence of other carbohydrates in the functional food product. Isomaltulose acts in this use according to the invention as a modulator or influencing variable on the regeneration, caused by other carbohydrates, of the body composition, in particular the RQ of physically exerted individuals.

In a further preferred embodiment, the invention relates to the present uses, wherein the respiratory quotient corresponding to the improved regeneration is present 0 to 24, preferably 0 to 12, in particular 0 to 4, preferably 0 to 2 hours after completion of the physical exertion and the consumption of the isomaltulose-containing food product, the term “0 hours” referring in particular to at least one minute or at least a plurality of minutes. In a particularly preferred embodiment, the respiratory quotient which is obtained in accordance with the invention and corresponds to the improved regeneration is obtained not just in the short term, but rather in the short and in the long term, preferably in the long term, for example for a period of from 3 to 24, 4 to 24, 5 to 24 or 6 to 24 hours after completion of the physical exertion and the ingestion of food taking place immediately thereafter, i.e. shortly thereafter.

The term “0 hours after exertion” refers to the fact that the respiratory quotient is present immediately after completion of the physical exertion, i.e. not at the same time, but rather immediately thereafter, for example one minute or a few minutes thereafter up to for example 24 hours after the end of the physical exertion.

In a further preferred embodiment, the present invention relates to the present uses, wherein the respiratory quotient which is present immediately prior to the physical exertion is present from 60 to 0, preferably 60 to 1 minutes, in particular 30 to 0 minutes, preferably 30 to 1 minutes prior to the start of the physical exertion.

In a further preferred embodiment, the present invention relates to the present use, wherein the physical exertion corresponds to a consumption of energy of from 0.02 to 0.5 kcal/kg of body weight/minute.

The present invention relates in a preferred manner to the aforementioned uses, wherein the individual is a human being or an animal, in particular a mammal, preferably a human being.

The present invention relates in a further preferred embodiment to a food product which is present in the form of a liquid food product, for example a nutrition solution or a drink, a solid food product or a semi-solid food product.

In a further preferred embodiment, the food product is a soft drink, a fruit juice drink, an enteral nutrition solution, a hypotonic drink, an isotonic drink, a hypertonic drink, an energy drink, a tea drink, a coffee drink, a sports drink, a cocoa drink, an energy drink, a milk drink or a drink powder.

In a further preferred embodiment, the food product is an energy bar, a muesli product, a milk product, a dairy product, a luxury food or a bakery product.

In a further preferred embodiment, the concentration of isomaltulose in the food product is from 1% to 99.9%, preferably from 1, 2, 3, 4, 5, 6, 7, 8, 9, 10, 20, 30, 40, 50, 60, 70, 80 or 90 to 99% by weight, in particular from 20 to 90, 30 to 80 or 40 to 70% by weight (based in each case on dry substance).

The isomaltulose can in one embodiment also be present in the form of a mixture. In connection with the present invention, in a particularly preferred embodiment the term “mixtures of isomaltulose” refers to the fact that the isomaltulose can be present in mixtures with suitable further substances, for example starch, starch derivatives, dextrins, for example nutriose, inulin, fructooligosaccharides, leucrose or trehalose. In a particularly preferred embodiment, in a mixture of this type the isomaltulose is present in a quantity of from 30 to 70, preferably 40 to 60% by weight and the at least one mix component is present in a quantitative ratio of 70 to 30, preferably 60 to 40% by weight (in each case dry substance based on the mixture). Mixtures of this type can in a preferred embodiment contain for example 1 to 20% by weight of isomaltulose and 80 to 99% by weight of other substances, for example other carbohydrates or intensive sweeteners. Mixtures of this type can however also contain 70 to 99% by weight of isomaltulose and 1 to 30% by weight of other substances, for example carbohydrates or intensive sweeteners. Obviously, however, the invention also includes other mixture ratios of isomaltulose and other substances, for example carbohydrates or intensive sweeteners, for example from 20 to 70% by weight of isomaltulose and 30 to 80% by weight of other substances.

In a further preferred embodiment, the present invention relates to a present mixture containing isomaltulose, this mixture being free from sucrose, being free from glucose, being free from lactose, being free from fructose, being free from sorbitol, being free from xylitol, being free from mannitol or being free from one or more or all of the aforementioned sugars or sugar alcohols.

In a particularly preferred embodiment, the isomaltulose is the one and only body-imparting sweetening agent occurring in the functional food product. In a further preferred embodiment, the invention relates to an aforementioned functional food product, wherein isomaltulose is the one and only sugar occurring in the functional food product.

In connection with the present invention, the term “sweetening agent” refers to substances which have sweetening power and are added to for example foods or drinks to produce a sweet taste. In connection with the present invention, the sweetening agents are divided into sugars such as isomaltulose, sucrose, glucose or fructose, which impart body and sweetening power, and sweeteners, i.e. substances which are not sugars, but nevertheless have sweetening power, and wherein said sweeteners are in turn subdivided into sugar substitutes, i.e. sweetening agents which have a body and a physiological calorific value in addition to a sweetening power, and intensive sweeteners, i.e. substances which generally have a very high sweetening power, but no body and generally no or only a minor physiological calorific value.

In a further preferred embodiment, the aforementioned functional food product is suitable for the specific nutrition of sportsmen, overweight persons, obese persons, diabetics or elderly persons.

In a further preferred embodiment, the invention provides the use of isomaltulose or mixtures of isomaltulose for the production of functional food products for improving the effect of physical exertion on individuals.

In a further preferred embodiment, the present invention relates to the use of isomaltulose or mixtures of isomaltulose for the production of functional food products for achieving a physical training effect on individuals.

Obviously, in addition to the aforementioned use aspects, the invention also relates to the corresponding method teachings, namely to methods for improving the regeneration of individuals exposed to physical exertion, isomaltulose or mixtures of isomaltulose being supplied to these individuals in the form of functional food products, in particular after completion of the physical exertion, preferably immediately on conclusion. Further advantageous method aspects emerge from the aforementioned use aspects.

Further advantageous embodiments emerge from the sub-claims.

The invention will be described in greater detail with reference to the following examples and the figures pertaining thereto.

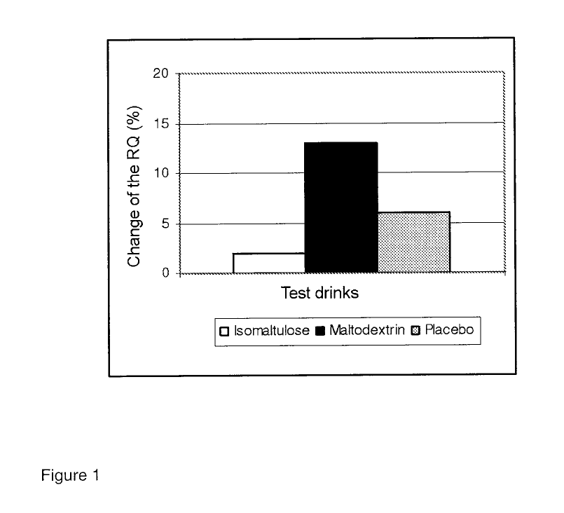

FIG. 1 shows the percentage change of the respiratory quotient (RQ) after consumption of drinks without carbohydrates, with isomaltulose and with maltodextrin.

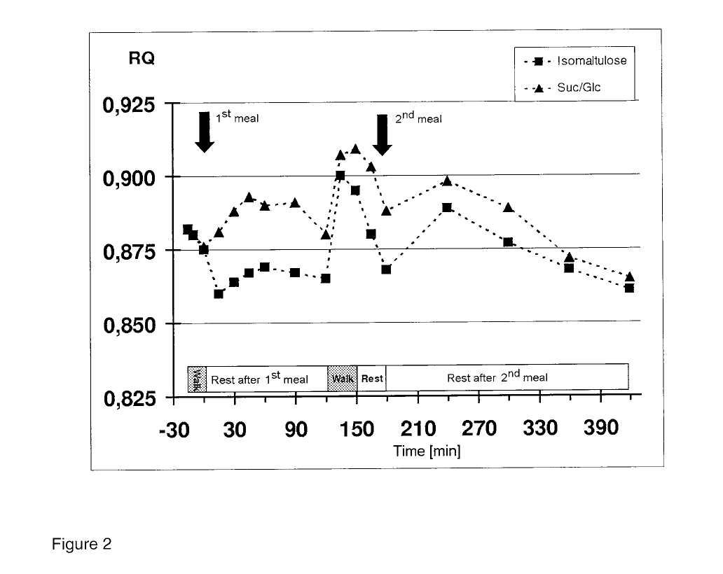

FIG. 2 shows the course of the RQ over a test period including two meals.

### BIBLIOGRAPHY

1. Dionne et al., Am J Clin Nutr (1999) 69, 927-30.
2. Saris et al., Int J Obes Relat Metab Disoerd (2004) 28(6), 759-65.
3. Tittelbach et al., Obes Res (2000) 8, 496-505.
4. Achten & Jeukendrup, J Sports Sci (2003) 21, 1017-1024
5. Schneiter et al., Am J Physiol (1995) 269, E1031-6
6. Brand-Miller et al., Am J Clin Nutr (2002) 76, 281-5
7. Burke et al., J. Appl. Physiol. (1998) 85, 2220-2226

### EXAMPLE 1

The effect of isomaltulose (Palatinose™) on the regulation of the metabolism before, during and after physical exertion was examined and compared with the consumption of maltodextrin or a placebo which has a non-glycaemic effect and is of equivalent sweetness.

Isomaltulose formulation

| Item | Raw material | Quantity % |
|---|---|---|
| 1 | Isomaltulose | 93.98 |
| 2 | Citric acid (anhydrous) | 2.50 |
| 3 | 311744 Eurocert Quinoline Yellow dye | 0.004 |
| 4 | Lemon flavouring agent 210336 from Symrise | 1.00 |
| 5 | Sucralose | 0.03 |
| Sum | | 100.00 |

Maltodextrin formulation

| Item | Raw material | Quantity % |
|---|---|---|
| 1 | Maltodextrin, 2022225 from Agrana | 93.96 |
| 2 | Citric acid (anhydrous) | 2.50 |
| 3 | 311744 Eurocert Quinoline Yellow dye | 0.004 |
| 4 | Lemon flavouring agent 210336 from Symrise | 1.00 |
| 5 | Sucralose | 0.05 |
| Sum | | 100.00 |

Placebo formulation (inulin-based)

| Item | Raw material | Quantity % |
|---|---|---|
| 1 | Inulin RAFTILINE ST gel (instant) | 64.46 |
| 2 | Citric acid (anhydrous) | 25.00 |
| 3 | 311744 Eurocert Quinoline Yellow dye | 0.04 |
| 4 | Lemon flavouring agent 210336 from Symrise | 10.00 |
| 5 | Sucralose | 0.50 |
| Sum | | |

The test was carried out as a 3-armed crossover study on 21 subjects. In each subject, the effect of a non-glycaemic drink (placebo=sweetened mineral water) was tested versus a drink containing maltodextrin versus isomaltulose.

The subjects were male endurance athletes with several years' training experience (VO2max>55 ml/kg KG) aged between 23 and 50.

In step ergometry on a bicycle (100 watts at the start; increased by 50 watts/3 min), performance was first determined in the range of from 70-75% of the maximum oxygen uptake. After a sufficient regeneration phase of at least 5 days, the first testing was carried out in randomised order. The testing was carried out in the morning, at the same time in each case, 3-4 hours after a standardised breakfast.

Standardised endurance exertion was first carried out on a bicycle ergometer at 70-75% of the VO2max over 90 min. An anaerobic Wingate test immediately followed the 90-minute submaximum exertion. 250 ml of the respective drink with 25 g isomaltulose or maltodextrin or without carbohydrates were supplied 30 min prior to the exertion, immediately at the start of the exertion, after 45 min of the endurance exertion and immediately after completion of the Wingate test (in this case now as 2×250 ml). The respiratory quotient (RQ) was determined at the moments −30, −15, 0, 15, 30, 45, 60, 75, 90 min before or during the exertion, immediately after completion of the Wingate test and also 3, 15, 30, 60 and 120 min after end of exertion. Further tested parameters included glucose, lactate, free fatty acids, insulin.

The statistical evaluation was carried out using SPSS Version 13.1. The data was checked for significant differences (as a function of the drink supplied) by means of the Wilcoxon test for paired data. A p value of <0.05 was deemed to be significant. The multiple testing was taken into account by way of Holm correaction.

Throughout the test phase, the supply of isomaltulose was followed by a lower RQ. In the case of the isomaltulose drink, the RQ was surprisingly substantially equal after the physical exertion to the quotient immediately prior to the exertion. Conversely, the respiratory quotient after the physical exertion was in the case of the maltodextrin drink higher by 13% and in the case of the placebo higher by about 6% than immediately prior to the exertion. This is also shown in the figure which shows the percentage changes of the RQ values after versus prior to the physical exertion.

### EXAMPLE 2

### Recipe for an Orange Drink

### Isomaltulose Instant Orange Drink Containing L-Carnitine

| Item | Ingredients | Content |
|---|---|---|
| 1 | Isomaltulose | 92.60% |
| 2 | Citric acid (anhydrous) | 4.96% |
| 3 | Trisodium citrate | 0.26% |
| 4 | Tricalcium phosphate | 0.22% |
| 5 | Vitamin C | 0.24% |
| 6 | Opacifier containing E 171 dye | 0.48% |
| 7 | E 102 dye (85%) | 0.01% |
| 8 | E 110 dye (85%) | 0.016% |
| 9 | E 414 gum arabic (spray-dried) | 0.10% |
| 10 | E 415 xanthan | 0.10% |
| 11 | E 466 Na carboxymethylcellulose | 0.10% |
| 12 | Type 100 orange flavouring agent | 0.64% |
| 13 | Type 120 orange flavouring agent | 0.24% |
| 14 | Sucralose | 0.03% |
| Sum | | 100.0% |

### EXAMPLE 3

### Recipe for a Sports Drink

### Isomaltulose Sports Drink Containing L-Carnitine

| Item | Ingredients | Content |
|---|---|---|
| 1 | Isomaltulose | 90.04% |
| 2 | Citric acid (anhydrous) | 6.360% |
| 3 | Vitamin C | 0.550% |
| 4 | Trisodium citrate | 1.194% |
| 5 | Opacifier containing E 171 dye | 0.262% |
| 6 | E 415 xanthan | 0.091% |
| 7 | E 466 Na carboxymethylcellulose | 0.091% |
| 8 | Sucralose | 0.300% |
| 9 | E 102 dye (85%) | 0.018% |
| 10 | Grapefruit lemon flavouring agent | 1.090% |
| Sum | | 100.00% |

### EXAMPLE 4

### Recipe for an ACE Drink

### Isomaltulose ACE Drink

| Item | Ingredients | Content |
|---|---|---|
| 1 | Isomaltulose | 94.52% |
| 2 | Citric acid (anhydrous) | 3.88% |
| 3 | Trisodium citrate | 0.27% |
| 4 | Tricalcium phosphate | 0.25% |
| 5 | Opacifier containing E 171 dye | 0.30% |
| 7 | E 110 dye (85%) | 0.033% |
| 8 | E 102 dye (85%) | 0.0125% |
| 9 | Coffee brown TF 8 dye | 0.0025% |
| 10 | E129 (Allura red) dye | 0.0015% |
| 11 | Vitamin E | 0.02% |
| 12 | Provitamin A | 0.032% |
| 13 | E 415 xanthan | 0.170% |
| 14 | E 466 Na carboxymethylcellulose | 0.170% |
| 15 | Multiple-fruit flavouring agent | 0.330% |
| Sum | | 100.00% |

### EXAMPLE 5

### Second Meal Effect

The test was a crossover study with 20 subjects. In each subject, the effect of isomaltulose vs. sucrose/glucose syrup was tested.

The test period comprised two meals into which the test substances were integrated: on the one hand a breakfast, which consisted of a drink (250 ml, 10% carbohydrates=25 g of test substance) and biscuits (approx. 140 g, also with 25 g of test substance), and on the other hand a lunch consisting of mini pizzas, an apple and a refreshment drink. The carbohydrate quantity, consumed per portion of shortpastry, of wheat starch of the flour was, at about 50-60 g carbohydrates, at the same level in all biscuit variants. Each test period comprised two postprandial phases.

The respiratory quotient was determined first at rest, then during the subsequent 30-min exertion at moderate intensity (treadmill uphill protocol 4 km/h 5% gradient), at the end of the 30-minute post-exertion phase and up to 4 hours after consumption of the second meal (the lunch).

The RQ after consumption of the isomaltulose breakfast fell and was lower than after the sucrose/glucose syrup breakfast, while at the same time the degree by which the RQ was lower was maintained even after consumption of a second meal (lunch). The RQ curves extended immediately after the initial reduction of the RQ through the isomaltulose breakfast parallel up to the end of the test period. The RQ set point determines the RQ over the entire day.

1-20. (canceled)

21. A method for improving the regeneration of individuals exposed to physical exertion, wherein the improved regeneration is indicated by a respiratory quotient which is present after exertion and which is substantially equal to or lower than a respiratory quotient of the same individual that is present immediately prior to the physical exertion, said method comprising consumption by the individual exposed to said physical exertion of a functional food product comprising at least one of isomaltulose and mixtures containing isomaltulose, wherein said food product is intended and suitable for consumption after completion of physical exertion.

22. The method according to claim 21, wherein the improved regeneration is indicated by a respiratory quotient of said individual which, upon ingestion of said functional food product containing said at least one of isomaltulose and mixtures of isomaltulose, is lower compared to that obtained upon ingestion by said individual of a food product otherwise identical to said functional food product but containing other carbohydrates without said at least one of isomaltulose and mixtures containing isomaltulose.

23. The method according to claim 22, wherein said other carbohydrates are high-glycaemic carbohydrates.

24. The method according to claim 21, wherein the improved regeneration is indicated by a respiratory quotient of said individual which, upon ingestion of said functional food product containing said at least one of isomaltulose and mixtures containing isomaltulose, is lower compared to that obtained upon ingestion by said individual of a food product otherwise identical to said functional food product but containing no carbohydrates.

25. The method according to claim 21, wherein the respiratory quotient corresponding to the improved regeneration is present at from 0 to 24 hours after the physical exertion.

26. The method according to claim 25, wherein the respiratory quotient corresponding to the improved regeneration is present at from 0 to 4 hours after the physical exertion.

27. The method according to claim 21, wherein the respiratory quotient that is present immediately prior to the physical exertion is substantially the same as the respiratory quotient that is present at from 60 to 0 minutes prior to the physical exertion.

28. The method according to claim 21, wherein the physical exertion corresponds to a consumption of energy of from 0.02 to 0.5 kcal/kg of body weight/minute.

29. The method according to claim 21, wherein the individual is a human being.

30. The method according to claim 21, wherein the food product is selected from the group consisting of drinks, semi-solid food products and solid food products.

31. The method according to claim 21, wherein the food product is selected from the group consisting of a soft drink, a fruit juice drink, an enternal nutrition solution, a hypotonic drink, an isotonic drink, a hypertonic drink, an energy drink, a tea drink, a coffee drink, a sports drink, a cocoa drink, an energy drink, a milk drink and a drink powder.

32. The method according to claim 21, wherein the food product is selected from the group consisting of an energy bar, a muesli product, a milk product, a dairy product, a luxury food and a bakery product.

33. The method according to claim 21, wherein the functional food product contains a concentration of isomaltulose of from 1% to 99% by weight, based on weight of dry substance.

34. The method according to claim 21, wherein the functional food product is suitable and intended to be consumed in the form of a first meal of two meals at different times after completion of the physical exertion and wherein the improved regeneration is indicated by a respiratory quotient which is present after exertion and consumption of both meals and is substantially equal to or lower than a respiratory quotient of the same individual that is present immediately prior to the physical exertion, and wherein at least the food product consumed in the form of said first meal is intended and suitable for consumption after completion of the physical exertion.

35. The method according to claim 34, wherein the second meal contains isomaltulose.

36. The method according to claim 34, wherein the second meal is substantially free of isomaltulose.

37. The method according to claim 21, wherein the food product is suitable for specific nutrition of at least one said individual selected from the group consisting of sportsmen, overweight persons, obese persons diabetics and elderly persons.

38. A method of making a functional food product which reduces the effect of physical exertion upon an individual consuming said food product, the method comprising preparing a food product intended and suitable for consumption by said individual after completion of the physical exertion, wherein the food product comprises at least one of isomaltulose and mixtures containing isomaltulose.

39. A method of making a functional food product which achieves a physical training effect upon an individual consuming said food product, the method comprising preparing a food product intended and suitable for consumption by an individual undergoing physical training, wherein the food product comprises at least one of isomaltulose and mixtures containing isomaltulose.

40. A method of making a functional food product which achieves a second meal effect upon an individual consuming said food product, the method comprising preparing a food product intended and suitable for consumption by an individual desiring to obtain said second meal effect, wherein the food product comprises at least one of isomaltulose and mixtures containing isomaltulose.

41. The method according to claim 31, wherein said mixtures contain, in addition to isomaltulose, at least one of carbohydrates and intensive sweeteners.
